(19) United States
(12) Patent Application Publication
(10) Pub. No.: US 2026/0248618 A1
(43) Pub. Date: Aug. 27, 2026

(54) REPLACEMENT ARTHROPLASTY AND JOINT RESURFACING ARTHROPLASTY IMPLANT WITH NON-STEMMED FIXATION, METHOD OF IMPLANTATION, AND METHOD OF USING SAME

(71) Applicant: Eduardo Gonzalez-Hernandez, Miami, FL (US)

(72) Inventor: Eduardo Gonzalez-Hernandez, Miami, FL (US)

(21) Appl. No.: 19/546,220

(22) Filed: Feb. 20, 2026

Related U.S. Application Data
(60) Provisional application No. 63/761,533, filed on Feb. 21, 2025.

(51) Int. Cl. A61F 2/42; A61B 17/17; A61B 17/56

(52) U.S. Cl. CPC A61F 2/4261; A61B 17/1782; A61B 2017/564; A61F 2002/4264

(57) ABSTRACT

The present disclosure provides an implant, a corresponding method of implantation, and a method of using the same that can be used in orthopaedic joint replacement arthroplasty or hemiarthroplasty and/or an orthopaedic joint resurfacing arthroplasty or hemiarthroplasty. The implant can include a first fin portion and a second fin portion for receipt in a first slot and a second slot, respectively formed in bone, and the first fin portion and the second fin portion can support an articulation portion that can be used in resurfacing and replacing portions of articulation surfaces of a joint.

The present application claims the benefit of U.S. Provisional Application No. 63/761,533, filed Feb. 21, 2025; all of which is incorporated by reference herein.

### FIELD

The present disclosure relates to an implant, a corresponding method of implantation, and a method of using the same that can be used in orthopaedic joint replacement arthroplasty or hemiarthroplasty and/or an orthopaedic joint resurfacing arthroplasty or hemiarthroplasty. The implant of the present disclosure can be used for surgical reconstruction, for example, of a musculoskeletal joint such as a radiocarpal joint, an ankle joint, a knee joint, etc.

### BACKGROUND

Arthritic degeneration or deformation of a joint may result from an inflammatory condition such as rheumatoid arthritis or ligamentous instability. Furthermore, a joint may degenerate or deform as a result of trauma such as intra-articular fracture or other post-traumatic injury. To treat such degeneration or deformation, conventional replacement arthroplasty/hemiarthroplasty and joint resurfacing arthroplasty/hemiarthroplasty typically employ a conventional device in the form of a stemmed intermedullary implant including an intramedullary stem that requires extensive exposure of the joint, including joint dislocation, to facilitate excision of a substantial amount of bone for implantation of the intramedullary stem. An articular component of the conventional intermedullary stemmed implant can be mated to the intramedullary stem either as a single piece (monoblock) or as a modular assembly.

Such a conventional intermedullary stemmed implant is generally indicated by the numeral 100 in FIG. 11. The conventional intermedullary stemmed implant 100 of FIG. 11 includes an articular surface 102 and a intermedullary stem 104, and use thereof has multiple limitations. Limitations exist due to the intermedullary stem 104 needing to tightly fit in a medullary canal for secure attachment, and adjustments of the articular surface 102 being correspondingly limited thereby. Limitations also exist because a substantial amount of bone excision may be required to facilitate attachment of the conventional intermedullary stemmed implant 100.

To illustrate, the conventional intermedullary stemmed implant 100 can be attached to a distal portion of a radius, and correspondingly, can be used in surgical reconstruction of a radiocarpal joint. The conventional intermedullary stemmed implant 100 can present a substantial imposition on local anatomy of the radiocarpal joint because the articular surface 102 may end up in a position limited and determined by the fitting of the intermedullary stem 104 in the medullary canal. Due to the position of the intermedullary stem 104, the articular surface 102 may resultantly be in a position that is too proud after implantation, because the intermedullary stem 104 is too big for the distal portion of the radius. Furthermore, an inclination of the articular surface 102 may result in an undesirable position that is not modifiable because of the configuration of the intermedullary stem 104.

As a result of above-discussed limitations, the present disclosure provides an improved joint replacement arthroplasty or hemiarthroplasty implant and/or a joint resurfacing arthroplasty or hemiarthroplasty implant, a corresponding method of implantation, and a method of using the same, where the fixation of articular surface to the bone is a departure from conventional implants employing a conventional intramedullary stem. Using the improved implant and the corresponding method of implantation, the fixation to the bone can be accomplished using one or more medullary fins or posts, the benefits of which will become apparent. The present disclosure also provides cutting guides and methods for use thereof that also address the above-discussed limitations. And while the present disclosure is focused on the radiocarpal joint, the implant and methods disclosed herein can be configured for use in other joints of the human body to improve (and potentially restore) joint function via an arthroplasty or hemiarthroplasty and/or a joint resurfacing arthroplasty or hemiarthroplasty.

### SUMMARY

An arthroplasty or hemiarthroplasty implant and a joint resurfacing arthroplasty or hemiarthroplasty implant, a corresponding method of implantation, and a method of using the same are provided in present disclosure, and can be used in orthopaedic joint replacement arthroplasty/hemiarthroplasty and/or an orthopaedic joint resurfacing arthroplasty/hemiarthroplasty.

In one aspect, the present disclosure provides a method of implanting an implant configured for joint replacement arthroplasty or hemiarthroplasty or configured for joint resurfacing arthroplasty or hemiarthroplasty, the method including accessing a radiocarpal joint through a dorsal portion of a human wrist; positioning a portion of a guide adjacent a dorsal portion of a distal portion of a radius of the radiocarpal joint; using the guide to create a first slot in the distal portion of the radius that extends from a distal end of the radius toward a proximal end of the radius; using the guide to create a second slot in the distal portion of the radius adjacent the first slot that extends from the distal end of the radius toward the proximal end of the radius positioning a first fin portion of the implant in the first slot, positioning a second fin portion of the implant in the second slot, and positioning an articulation portion of the implant into the radiocarpal joint, the first fin portion and the second fin portion extending outwardly from the articulation portion; positioning an articulation surface of the articulation portion adjacent the distal end of the radius; and improving function of the radiocarpal joint via interaction between the articulation surface of the articulation portion and proximal portions of one or more carpals adjacent the radiocarpal joint.

In another aspect, the present disclosure provides a method of implanting an implant configured for joint replacement arthroplasty or hemiarthroplasty or configured for joint resurfacing arthroplasty or hemiarthroplasty, the method including accessing a radiocarpal joint through a dorsal portion of a human wrist; positioning a first fin portion of the implant in a first slot formed in a distal portion of a radius, positioning a second fin portion of the implant in a second slot formed in the distal portion of the radius, and positioning an articulation portion of the implant into a radiocarpal joint corresponding to the radius, the first fin portion and the second fin portion extending outwardly from the articulation portion; positioning an articulation surface of the articulation portion adjacent the distal end of the radius; inserting a bone screw through an aperture formed in an end portion attached to the first fin portion and into the radius, the end portion being attached to the first fin portion opposite from the articulation portion; and improving function of the radiocarpal joint via interaction between the articulation surface of the articulation portion and proximal portions of one or more carpals adjacent the radiocarpal joint.

In yet another aspect, the present disclosure provides an implant configured for joint replacement arthroplasty or hemiarthroplasty or configured for joint resurfacing arthroplasty or hemiarthroplasty, the implant including an articulation portion having an articulation surface and an opposite undersurface; a first fin portion extending outwardly from the undersurface; a second fin portion extending outwardly from the undersurface; and a first end portion attached to the first fin portion opposite from the undersurface, the first end portion including an aperture therethrough facilitating receipt of an bone screw; where the first fin portion and the second fin portion are configured to fit in a first slot and a second slot, respectively, formed in a distal portion of a radius, and the articulation surface is configured to form portions of a radiocarpal joint to improve function thereof.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a front elevational view of an implant according to an embodiment of the present disclosure;

FIG. 2 is a first side elevational view of the implant of FIG. 1;

FIG. 3 is a front, second side perspective view of the implant of FIG. 1 positioned within a distal portion of a radius shown in phantom;

FIG. 4 is a dorsal, lateral perspective view of the distal portion of the radius with the implant of FIG. 1 implanted therein;

FIG. 5 is a dorsal, medial perspective view of the distal portion of the radius with the implant of FIG. 1 implanted therein;

FIG. 6 is a dorsal, medial perspective view of the distal portion of the radius with a cutting guide being positioned relative to a radiocarpal joint and adjacent a dorsal side of the radius;

FIG. 7 is a medial elevational view of the distal portion of the radius with the cutting guide of FIG. 6 being positioned relative to the radiocarpal joint;

FIG. 8 is a top plan view of the distal portion of the radius and adjacent ulna depicting a preferred flexion-extension axis of the radiocarpal joint;

FIG. 9 is a dorsal, medial perspective view of the distal portion of the radius, similar to FIG. 6, showing bending movement of a handle portion of the cutting guide of FIG. 6;

FIG. 10 is a dorsal, medial perspective view of the distal portion of the radius, similar to FIG. 6, showing twisting movement of the handle portion of the cutting guide of FIG. 6; and

FIG. 11 is a perspective view of a prior art intermedullary-stemmed implant.

### DETAILED DESCRIPTION

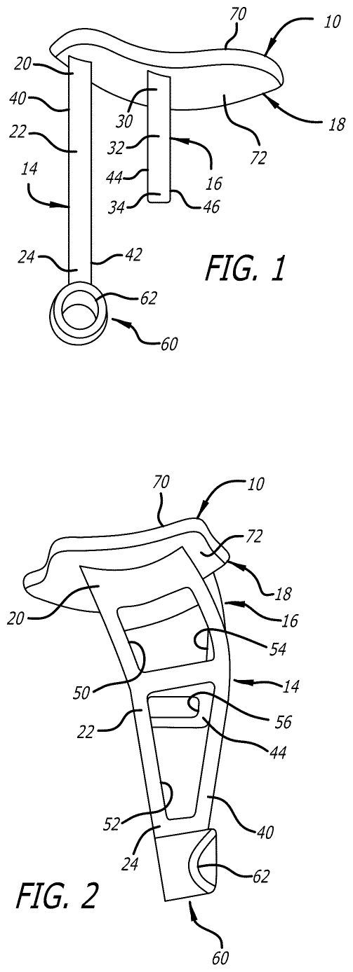

The present disclosure is directed to embodiments of an implant generally referenced by the number 10 in FIGS. 1-5, and methods for use thereof that can be used by a surgeon in an arthroplasty or hemiarthroplasty and/or a joint resurfacing arthroplasty or hemiarthroplasty. Such use of the implant 10, for example, can aid in reconstructing (via resurfacing and/or replacing) all or portions of a proximal or distal portion of a bone in a body of a patient, and such resurfacing and/or replacement can facilitate restoration of joint function of a corresponding joint and alleviation of discomfort and pain.

For example, the implant 10 is a prothesis that can be configured to resurface and/or replace all or portions of proximal or distal portions of a radius R to improve functionality of a corresponding joint. To illustrate, the implant 10 can be attached relative to a distal end portion or a proximal end portion of the radius R, and can be used in arthroplasty or hemiarthroplasty and/or a joint resurfacing arthroplasty or hemiarthroplasty of the distal ends thereof.

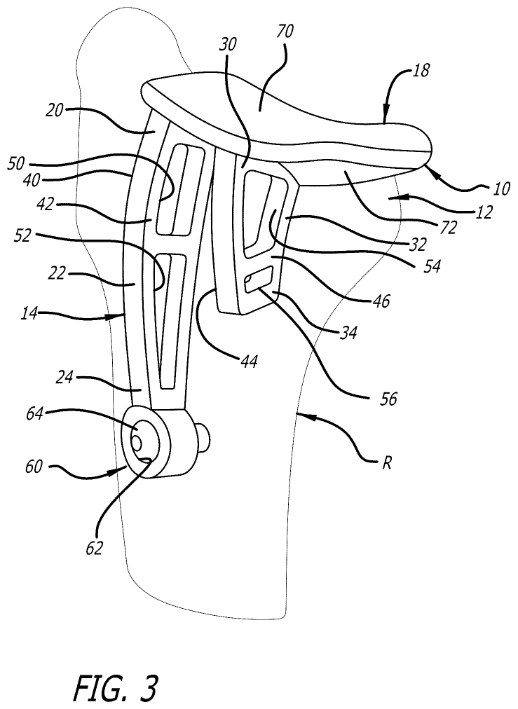

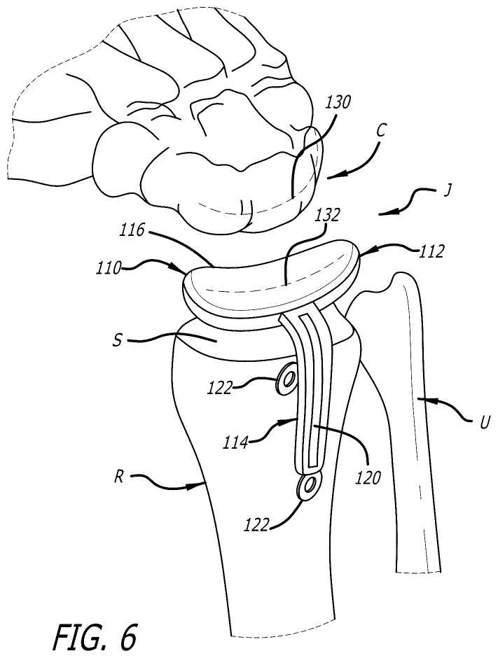

More specifically, as depicted in FIGS. 3-5, the implant 10 can be used for partial or total wrist arthroplasty replacement via resurfacing and/or replacement of an articular surface S (FIGS. 6, 9, and 10) of a distal end 12 of the radius R, and such resurfacing and/or replacement can improve the functionality of a radiocarpal joint J by, for example, improving extension and flexion of the radiocarpal joint J. In doing so, the implant 10 can be used in reproducing a patient's preferred flexion-extension wrist axis that can actually match or substantially match the patient's physiologic flexion-extension wrist axis. Additional benefits of use of the implant 10 include potentially less bone resection, potentially less need for dislocation maneuvering for joint exposure and access, preservation of joint capsule and joint ligaments, and more ideal and better implant positioning that can either better match local anatomy or modify the local anatomy in a way that is advantageous. In doing so, the implant 10 can extend a replacement articulation surface in an ulnar direction to better align a corresponding carpus on the forearm.

Different sizes of the implant 10 can be provided to accommodate anatomies of differently-sized patients. The implant 10 can be implanted via attachment to the distal portion of the 12 radius R, and portions of the implant 10 can fill portions of the radiocarpal joint J. As such, the implant 10 can interact with proximal portions of one or more carpals (e.g., a scaphoid and/or a lunate) at and adjacent the radiocarpal joint J, and can potentially result in adduction and tightening of the radiocarpal joint capsule and radiocarpal joint ligaments of the radiocarpal joint J.

As discussed below, the implant 10 can include one or more fin portions that are attached to an articulation (or plate) portion, and the fin portion(s) can be attached to distal or proximal end portions of bone (such as the distal end portion of the radius R) to support the articulation portion relative thereto. Furthermore, the articulation portion can include one or more articulation surfaces used for positioning adjacent a joint (such as the radiocarpal joint J), and the articulation surface(s) can serve as a prosthetic articular portion that facilitates resurfacing and/or replacement of the distal or proximal ends (such as the distal end 12) to facilitate cooperation with adjacent bones (e.g., the scaphoid and the lunate) during extension and flexion of the joint (such as the radiocarpal joint J).

As depicted in FIGS. 1-3, the implant 10 includes a first fin (or post) portion 14, a second fin (or post) portion 16, and an articulation (or plate) portion 18. For example, each of the first fin portion 14 and the second fin portion 16 can be attached to the distal end portion of the radius R, and the articulation portion 18 can resurface and/or replace the distal end 12 of the radius R and correspondingly interface with the proximal portions of the scaphoid and/or the lunate. The first fin portion 14 and the second fin portion 16 can serve as attachment lattices for securing the implant 10 to bone, and portions the articulation portion 18 can serve as a prothesis replacing portions of the radiocarpal joint J. While the implant 10 is attached to the distal end 12 of the radius R, and positioned relative to the, the present disclosure is not so limited. The implant 10 can be configured for and used with distal ends of other bones and correspondingly positioned relative to other joints of the human body to improve (and potentially restore) joint function.

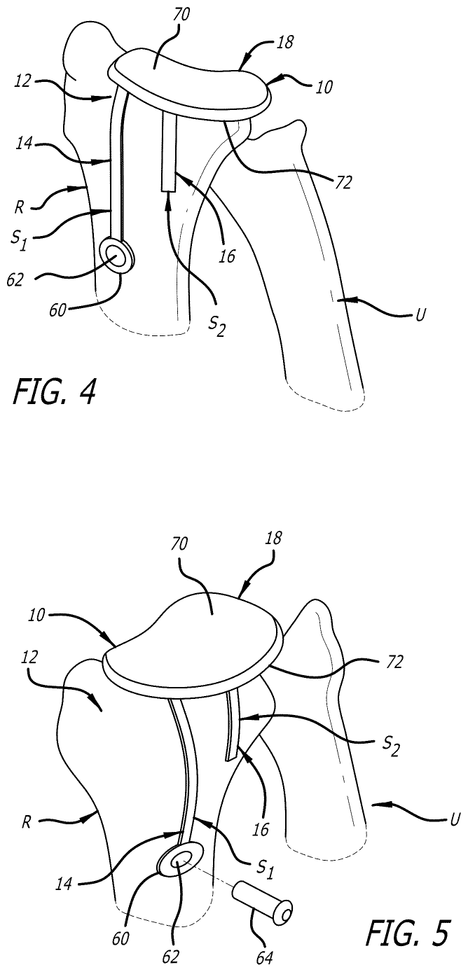

Either of the first fin portion 14 or the second fin portion 16 can be an at least partially intramedullary component, and the articulation portion 18 can be an extra-medullary component. For example, during implantation of the implant 10, the first fin portion 14 is configured to fit within a cortical defect such as a first slot (notch or slit) S₁ (FIGS. 4 and 5) formed in the radius R, and the second fin portion 16 is configured to fit within a cortical defect such as a second slot (notch or slit) S₂ formed in the radius R. The first fin portion 14 and the second fin portion 14 can be attached to the subchondral bone adjacent the radiocarpal joint J. The first fin portion 14 includes a proximal portion 20, an intermediate portion 22, and a distal portion 24, and the second fin portion 16 includes a proximal portion 30, an intermediate portion 32, and a distal portion 34.

As depicted in FIGS. 1-3, the first fin portion 14 can include a first side surface 40 and a second side surface 42, which extend over all or portions of the proximal portion 20, the intermediate portion 22, and the distal portion 24, and the second fin portion 16 can include a first side surface 44 and a second side surface 46, which extend over all or portions of the proximal portion 30, the intermediate portion 32, and the distal portion 34. The first fin portion 14 can have a uniform or varied thickness between the first side surface 40 and the second side surface 42, and the second fin portion 16 can have a uniform or varied thickness between the first side surface 44 and the second side surface 46. The thicknesses of the first fin portion 14 and the second fin portion 16 can be sized to fit within the the slots S₁ and S₂. All or portions of the first side surfaces 40 and 44 and the second side surfaces 42 and 46 can be flat or non-flat, and/or textured or non-textured. To illustrate, the first side surfaces 40 and 44 and/or the second side surfaces 42 and 46 could be curved, include protrusions, include indentations, and/or together form a particular shape (e.g., T-shape, S-shape, or Y-shape). And the thicknesses of the first fin portion 14 and the second fin portion 16, and configurations of the first side surfaces 40 and 44 and the second side surfaces 42 and 46 can facilitate interference fits in the respective slots S₁ and S₂, and roughened texturing thereof can facilitate bony ingrowth thereinto.

As depicted in FIGS. 2 and 3, the first fin portion 14 and the second fin portion 16 can each include one or more apertures therethrough that can facilitate bone growth between opposite sides of the slots S₁ and S₂. For example, the first fin portion 14 can include an aperture 50 and an aperture 52 extending therethrough between the first side surface 40 and the second side surface 42, and the second fin portion 16 can include an aperture 54 and an aperture 56 extending therethrough between the first side surface 44 and the second side surface 46. The apertures 50, 52, 54, and 56 can be filled with bone-growth promoting substances, and after receipt of the first fin portion 14 and the second fin portion 16 in the slots S₁ and S₂, respectively, the bone-growth promoting substances can stimulate bone growth through the apertures 50, 52, 54, and 56 between the opposite sides of the slots S₁ and S₂. Furthermore, a mesh or mesh-like material (not shown) can be provided within and attached between sidewalls of the apertures 50, 52, 54, and 56, and the mesh or mesh-like material can serve as a lattice (with or without use of the bone-growth promoting substances) to facilitate the bone growth between the opposite sides of the slots S₁ and S₂.

Such bone growth through the apertures 50, 52, 54, and 56, as well as the above-discussed interference fits and bone ingrowth into the roughened texturing of the sides 40, 42, 44, can serve in securing attachment of the first fin portion 14 and the second fin portion 16 in position within the slots S₁ and S₂, respectively, and relative to the radius R. The receipt of the first fin portion 14 and the second fin portion 16 within the slots S₁ and S₂, respectively, can minimize the need for additional fixation, and such receipt and/or the other attachment mechanisms discussed below can provide for cementless attachment of the implant 10 to bone.

In addition, one or bone-screw receiving apertures in portions of the first fin portion 14 and the second fin portion 16 can also be provided to receive corresponding bone screws that can also serve in attaching the first fin portion 14 and the second fin portion 16 to the radius R. For example, as depicted in FIGS. 1-3, the first fin portion 14 includes an end portion 60 at and adjacent the distal portion 24 thereof, and the end portion 60 includes and a bone-screw receiving aperture 62 extending therethrough. After positioning of the implant 10 relative to the radius R (with the first fin portion 14 and the second fin portion 16 received within the slots S₁ and S₂, respectively), a bone screw 64 can inserted through the aperture 62 and into the radius R to fastenably secure the first fin portion 14 in position.

Although not shown, a similar end portion and an aperture can be attached the second fin portion 16 to facilitate receipt of a bone screw to also fastenably secure the second fin portion 16 in position. Furthermore, additional apertures and surrounding structures similar to the end portion 60 can be provided in, adjacent, or along other portions of the first fin portion 14 and the second fin portion 16 at various distances from the articulation portion 18 for receiving bone screws.

The articulation portion 18 can be attached to and supported by the proximal portions 20 and 30 of the first fin portion 14 and the second fin portion 16, respectively. The articulation portion 18 includes an articulation surface 70 for orientation toward the radiocarpal joint J, and an opposite undersurface 72 to which the proximal portions 20 and 30 of the first fin portion 14 and the second fin portion 16, respectively, can be attached.

The articulation surface 70 can be configured to replace all or portions of the articular surface S of the distal end 12 of the radius R, and can be shaped accordingly. To that end, the articulation surface 70 can be shaped with concave and/or convex (if any) contours that can be mapped and matched to corresponding concavities and convexities (if any) of the articular surface S of the distal end 12 of the radius R. To illustrate, the articulation surface 70 can be concave, and the concavity can be preferably elliptical in shape to correspondingly give the articulation surface 70 a spoon-like shape. Furthermore, the articulation surface 70 can have a concave curvature in a first plane substantially parallel to the first fin portion 14 and the second fin portion 16, and a concave curvature in a second plane perpendicular to the first plane. And the ellipse/ellipsoid may be substantially elongated or nearly hemispheric to match the patient's anatomy. The resulting shape of the articulation surface 70 can be similar, substantially similar, and/or identical to portions of the distal end 12 of the radius R. Depending on the portions of the distal end 12 of the radius R to be replaced by the articulation portion 18, the articulation surface 70 can be corresponding enlarged or reduced in size to facilitate such replacement or even extension the articular surface S.

The undersurface 72 can be convex, and in preparation for implantation of the implant 10, a concave rasp (not shown) can be used to achieve minimal bone resection of the distal portion 12 of the radius R. The undersurface 72, like the first fin portion 14 and the second fin portion 74, can have the roughened texturing to promote bony ingrowth to facilitate secure attachment of the implant 10 to the distal portion 12 of the radius R.

After implantation of the implant 10, for example, the articulation surface 70 can correspondingly interface with the proximal portions of the scaphoid and/or the lunate across the radiocarpal joint J to facilitate cooperation therewith. Such cooperation can serve in restoring joint function via restoration of joint mobility during extension and flexion, and such restoration of joint function can serve in alleviating discomfort and pain. Furthermore, the implant 10 (and the articulation surface 70) can also be configured for use in other joints of the human body to improve (and potentially restore) joint function via an arthroplasty or hemiarthroplasty and/or a joint resurfacing arthroplasty or hemiarthroplasty in similar fashion to that described hereinabove.

The slot S₁ and the slot S₂ can be formed using one or more drill guides, and can be created preferentially along the axis of a bone diaphysis-metaphysis (or metaphysis only) adjacent to a joint surface to be resurfaced and/or replaced. The goal of the cortical defect(s) are to permit placement of a metaphyseal or metaphyseal diaphyseal fixation of a component or components (such as the above-discussed first fin portion 14 and second fin portion 16) sideways into the bone. As discussed above, the receipt of the fin portion 14 in the slot S₁ and receipt of the second fin portion 16 in the slot S₂ can minimize the need for additional fixation.

The slot S₁ and the slot S₂ can be formed via abrading, cutting, grinding, sawing, and/or other bone removal techniques applied on a dorsal side of the radius R to a dorsal bone cortex thereof. FIGS. 4 and 5 illustrate that the slot S₁ and the slot S₂ can each extend from a somewhat medial portion of the distal end portion of the radius to the distal end 12 of the radius R. The slot S₁ and the slot S₂ can extend far enough into the bone to provide access to portions of a medullary canal of the radius R. Furthermore, the slot S₁ and slot S₂ the can have widths thereacross that correspond to the thicknesses of the first fin portion 14 and the second fin portion 16 to form the above-discussed interference fits (or at least tight fits) therebetween. As such, the first fin portion 14 can be inserted via slidable movement into the slot S₁ with portions thereof received in portions of the medullary canal of the radius R, and the second fin portion 16 can be inserted via slidable movement into the slot S₂ with portions thereof received in portions of the medullary canal of the radius R.

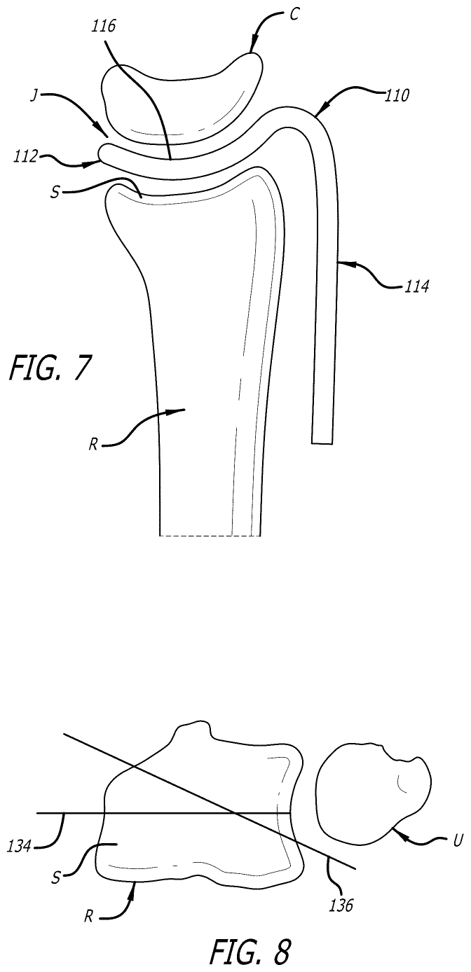

FIGS. 6, 9 and 10 depict use of a cutting guide 110 having a plate portion 112 and a handle portion 114 to facilitate placement of a permanent implant (such as the implant 10) for reconstructing all or portions of the articular surface S of the distal portion 12 of radius R of the radiocarpal joint J. As depicted in FIG. 7, the plate portion 112 of the cutting guide 110 is ultimately sandwiched between carpal bones C and the articular surface S of the distal portion 12 of the radius R of the radiocarpal joint J. Although the cutting guide 110 is configured to facilitate cutting of a single slot to afford placement of a corresponding permanent implant with a single fin portion, and the cutting guide 110 is not so limited. The cutting guide 110 can be repositioned or be modified to facilitate cutting two or more slots to afford placement of corresponding fins portions of a corresponding permanent implant with two or more fin portions. More specifically, the cutting guide 110 can be repositioned or modified to facilitate cutting of the slots S₁ and S₂ to afford placement of the implant 10 with the first fin portion 14 and the second fin portion 16.

The plate portion 112 of the cutting guide 110 includes an articular surface 116 that can be somewhat or virtually identical to that of the corresponding permanent implant. The handle portion 114 of the cutting guide 110 includes a slot (or slit) 120 for receiving a cutting instrument (not shown). During use, as depicted in FIGS. 6, 7, 9, and 10, the plate portion 112 of the cutting guide 110 is sandwiched in between the carpal bones C and the articular surface S of the distal portion 12 of the radius R to be resurface and/or replaced. With the wrist reduced and the articular surface 116 of the cutting guide 110 positioned between the carpal bones C and the articular surface S of the distal portion of the radius R, the surgeon can make the adjustments on the position of the cutting guide 110 to reproduce the patient's preferred flexion-extension wrist axis.

As depicted in FIG. 6, the long axis of the carpus for flexion and extension is illustrated with a dashed line 130. After proper positioning of the cutting guide 110, the long axis of the articular surface 116 that is illustrated with a solid line 132 in FIG. 6 should correspond to dashed line 130. FIG. 8 depicts a representation of the articular surface S of the radius R and an ulna U. A solid line 134 depicted in FIG. 5 suggests that the preferred flexion-extension axis of the radiocarpal joint J is along the long axis of the articular surface S of the radius R. However, the preferred axis of the wrist is the dart throwing axis represented by a solid line 136.

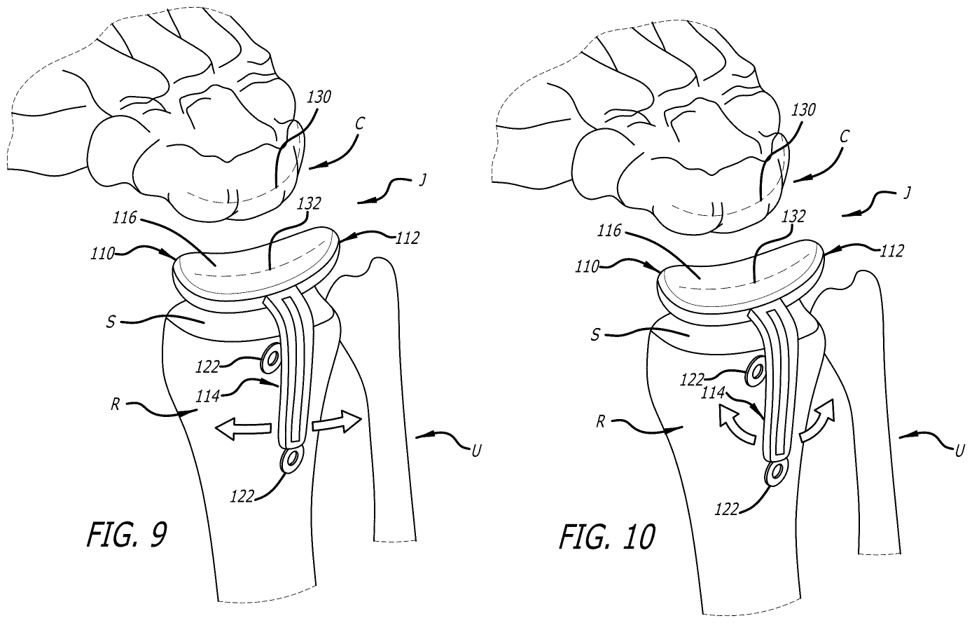

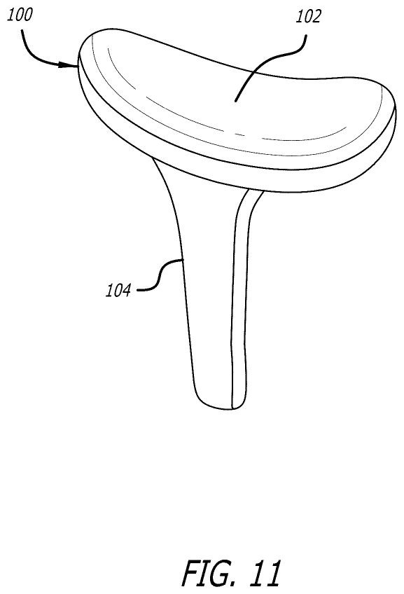

The cutting guide 110, as depicted in FIGS. 9 and 10, can be adjusted and moved about easily relative to the radiocarpal joint J during surgery to fit the preferred flexion-extension wrist axis that can actually match or substantially match the patient's physiologic flexion-extension wrist axis. The handle portion 114 of cutting guiding 110 may be swung (FIG. 9) or rotated (FIG. 10). The preferred flexion-extension wrist axis typically reproduces a dart throwing axis. To orient the articular surface 116 properly, the surgeon may flex and extend the patient's wrist several times until satisfied that the alignment of the articular surface 116 corresponds to the patient's preferred flexion extension axis. Such flexing and extending of the patient's wrist is not possible with current state of the art implants that use an intramedullary stem.

The surgeon has ample freedom for adjusting the articulating surface 116, and once satisfied with the proper position, the surgeon can then temporarily fix the cutting guide 110 to the radius R by using one or more K-wires (not shown) through K-wire apertures 122 provided on the handle portion 114. Thereafter, the surgeon can proceed to cut the corresponding slot or slots (for cutting guides with two or more fins) for the fin or fins which are part of the corresponding permanent implant (such as the implant 10). Once a slot or slots (for cutting guides with two or more fins) are made, the K-wire(s) are removed, the cutting guide 110 is removed, and the preparation of the distal portion of the radius R is made with a corresponding rasp or additional cutting features on the cutting guide 110. Thereafter, the corresponding permanent implant (such as the implant 10) can be implanted by sliding the fin or fins into the premade slot or slots, and insertion of bone screw(s) (such as the bone screw 64) into corresponding bone-screw receiving apertures (such as the aperture 62). The cutting guide 110, as depicted in FIGS. 6, 7, 9, and 10, is illustrated as being introduced through a dorsal approach. However, the cutting guide 110, can also be introduced through a ventral approach.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (for example, all described acts or events may not be necessary to carry out the techniques). In addition, while certain aspects of this disclosure are described as being performed by a single module or unit for purposes of clarity, it should be understood that the techniques of this disclosure may be performed by a combination of units or modules.

I claim:

1. A method of implanting an implant configured for joint replacement arthroplasty or hemiarthroplasty or configured for joint resurfacing arthroplasty or hemiarthroplasty, the method comprising:
accessing a radiocarpal joint through a dorsal portion of a human wrist;
positioning a portion of a guide adjacent a dorsal portion of a distal portion of a radius of the radiocarpal joint;
using the guide to create a first slot in the distal portion of the radius that extends from a distal end of the radius toward a proximal end of the radius;
using the guide to create a second slot in the distal portion of the radius adjacent the first slot that extends from the distal end of the radius toward the proximal end of the radius
positioning a first fin portion of the implant in the first slot, positioning a second fin portion of the implant in the second slot, and positioning an articulation portion of the implant into the radiocarpal joint, the first fin portion and the second fin portion extending outwardly from the articulation portion;
positioning an articulation surface of the articulation portion adjacent the distal end of the radius; and
improving function of the radiocarpal joint via interaction between the articulation surface of the articulation portion and proximal portions of one or more carpals adjacent the radiocarpal joint.

2. The method of claim 1, further comprising tightening a radiocarpal joint capsule and ligaments of the radiocarpal joint via receipt of the articulation portion in the radiocarpal joint capsule.

3. The method of claim 1, further comprising selecting from a selection of differently-sized implants, the implant that best fits anatomies of the radiocarpal joint.

4. The method of claim 1, wherein the guide is selected from a selection of differently-sized guides to best fit anatomies of the radiocarpal joint.

5. The method of claim 1, wherein each of the first slot and the second slot are created using abrading, cutting, grinding, sawing, and/or other bone removal techniques through at least one window provided in the guide.

6. The method of claim 5, wherein the at least one window includes dimensions corresponding to dimensions of the first fin portion and the second fin portion.

7. The method of claim 1, wherein each of the first slot and the second slot extend into a medullary canal of the distal portion of the radius.

8. The method of claim 1, wherein the articulation surface has a concave curvature in a first plane substantially parallel to the fin portion, and a convex curvature in a second plane perpendicular to the first plane.

9. A method of implanting an implant configured for joint replacement arthroplasty or hemiarthroplasty or configured for joint resurfacing arthroplasty or hemiarthroplasty, the method comprising:
accessing a radiocarpal joint through a dorsal portion of a human wrist;
positioning a first fin portion of the implant in a first slot formed in a distal portion of a radius, positioning a second fin portion of the implant in a second slot formed in the distal portion of the radius, and positioning an articulation portion of the implant into a radiocarpal joint corresponding to the radius, the first fin portion and the second fin portion extending outwardly from the articulation portion;
positioning an articulation surface of the articulation portion adjacent the distal end of the radius;
inserting a bone screw through an aperture formed in an end portion attached to the first fin portion and into the radius, the end portion being attached to the first fin portion opposite from the articulation portion; and
improving function of the radiocarpal joint via interaction between the articulation surface of the articulation portion and proximal portions of one or more carpals adjacent the radiocarpal joint.

10. The method of claim 9, further comprising tightening a radiocarpal joint capsule and ligaments of the radiocarpal joint via receipt of the articulation portion in the radiocarpal joint capsule.

11. The method of claim 9, wherein each of the first slot and the second slot extend into a medullary canal of the distal portion of the radius.

12. The method of claim 9, wherein the articulation surface has a concave curvature in a first plane substantially parallel to the fin portion, and a convex curvature in a second plane perpendicular to the first plane.

13. The method of claim 9, further comprising using a guide attached relative to the distal portion of the radius to form the first slot and the second slot.

14. The method of claim 13, wherein the guide is positioned in a first position to form the first slot, and positioned in a second position to form the second slot.

15. An implant configured for joint replacement arthroplasty or hemiarthroplasty or configured for joint resurfacing arthroplasty or hemiarthroplasty, the implant comprising:
an articulation portion having an articulation surface and an opposite undersurface;
a first fin portion extending outwardly from the undersurface;
a second fin portion extending outwardly from the undersurface; and
a first end portion attached to the first fin portion opposite from the undersurface, the first end portion including an aperture therethrough facilitating receipt of an bone screw;
wherein the first fin portion and the second fin portion are configured to fit in a first slot and a second slot, respectively, formed in a distal portion of a radius, and the articulation surface is configured to form portions of a radiocarpal joint to improve function thereof.

16. The implant of claim 15, wherein, after implantation of the implant, and the first fin portion and the second fin portion are respectively received in the first slot and the second slot, the bone screw can be received through the aperture of the end portion and into the radius.

17. The implant of claim 15, wherein at least one of the first fin portion and the second fin portion include an aperture formed therethrough to facilitate bone growth therethrough.

18. The implant of claim 17, wherein a mesh or mesh-like material is provided in the aperture to form a lattice facilitating bone ingrowth.

19. The implant of claim 15, wherein the first fin portion includes a first side surface, a second side surface, and a first thickness between the first and second side surfaces that correspond to a width of the first slot, and the second fin portion includes a third side surface, a fourth side surface, and a second thickness between the third and fourth side surfaces that correspond to a width of the second slot.

20. The implant of claim 19, wherein at least one of the first fin portion and the second fin portion include an aperture formed therethrough to facilitate bone growth therethrough, and a mesh or mesh-like material is provided in the aperture to form a lattice facilitating bone ingrowth.
